(19) United States
(12) Patent Application Publication
(10) Pub. No.: US 2022/0001297 A1
(43) Pub. Date: Jan. 6, 2022

(54) PROCESS FOR SELECTIVELY EXTRACTING CANNABINOIDS FROM CANNABIS PLANT MATERIALS

(71) Applicant: HEXO OPERATIONS INC., Ottawa (CA)

(72) Inventor: Max Alsayar, Ottawa (CA)

(21) Appl. No.: 17/294,858

(22) PCT Filed: Nov. 20, 2019
(86) PCT No.: PCT/CA2019/051659

§ 371 Date: May 18, 2021

Related U.S. Application Data
(60) Provisional application No. 62/769,800, filed on Nov. 20, 2018.

(51) Int. Cl. B01D 11/02

(52) U.S. Cl. CPC B01D 11/0203; B01D 11/0288; B01D 11/0292

(57) ABSTRACT

The present disclosure relates to a process for producing a cannabis concentrate from a cannabis plant material via extraction using supercritical carbon dioxide (CO₂) at temperatures in excess of 65° C., thus permitting the selective extraction of tetrahydrocannabinol (THC) over cannabidiol (CBD). Pressures in excess of 4200 psi (289.6 bar) also serve to further selectively extract THC over CBD.

### CROSS-REFERENCE TO RELATED APPLICATIONS

The present application claims the benefit of U.S. provisional patent application Ser. No. 62/769,800 filed on Nov. 20, 2018. The contents of the above-referenced document are incorporated herein by reference in their entirety.

### TECHNICAL FIELD

This application generally relates to the field of methods of extracting cannabinoids from cannabis plant materials.

### BACKGROUND

Cannabinoids have been used for many years, inter alia, in alleviating pain and inflammatory-related syndromes, spasms, asthma, sleep disorders, depression, loss of appetite and other medical conditions. The cannabinoids are a family of active compounds found mainly in the resin-producing pistillate inflorescences of cannabis plants. Although a variety of cannabinoid compounds have been identified in literature thus far, two compounds in particular have been the main focus of interest for medicinal and recreational uses: tetrahydrocannabinol (THC) and cannabidiol (CBD).

While THC is a psychoactive compound with adverse long-lasting effects on the user, CBD is not regarded as a psychotropic agent and is considered safe for consumption in various routes of administration. Depending on the cannabis plant strain both compounds can be typically found as a mixture, at various concentration ranges, in the plant source.

US 2008/0167483 describes a process for cannabinoid extraction from plant material using heat decarboxylation to convert cannabinoids in their acid forms to neutral forms, followed by CO₂ fluid extraction, and followed by ethanol winterization to remove waxes. This document teaches that contrary to expectations, it has determined that cannabinoids are best obtained under subcritical rather than supercritical CO₂ extraction conditions, namely best obtained with a temperature between 8-12° C., and a pressure between 55-65 bar (i.e., 800-950 psi).

WO 2018/061009 teaches that supercritical CO₂ extraction of cannabinoid species is often complicated, time consuming and very expensive compared to liquid extraction (e.g., ethanol extraction). WO 2018/061009 teaches that in addition, supercritical CO₂ extraction is far from being selective for specific cannabinoids, and may concomitantly extract also various essential oils.

A deficiency associated with the above methods, thus, lies in the low extraction yield and low (or no) selectivity. Namely, the extraction methods known to date extract various species of cannabinoids from the plant source, often resulting in an uncontrolled mixture of various concentrations and ratios of desired cannabinoids. Furthermore, the extracted cannabinoid extracts often have a bad taste associated therewith, likely due to the presence of residual winterization solvents and/or presence of bitter-tasting molecules, or chlorophyll or contaminants, which requires the addition of taste masking compounds to finished formulation for use in products for oral consumption. At least some of these deficiencies hinder subsequent formulation and use of cannabinoids in specific applications, such as for example, edibles, pharmaceuticals, beverages, vaping, and the like.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to an improved process for producing a cannabis concentrate enriched in a desired cannabinoid from a cannabis plant material, comprising varying process parameters in order to control a selective extraction of the desired cannabinoid.

As embodied and broadly described herein, the present disclosure relates to a process for extracting a cannabis concentrate from a cannabis plant material, the process comprising providing the plant material, the plant material having a first cannabinoid profile, and extracting the cannabis concentrate from the plant material using supercritical CO₂, the cannabis concentrate having a second cannabinoid profile wherein the first and second profiles are different, and wherein the extracting includes performing supercritical CO₂ extraction at a temperature >65° C. for selectively extracting tetrahydrocannabinol (THC) over cannabidiol (CBD) from the plant material.

As embodied and broadly described herein, the present disclosure relates to a process for extracting tetrahydrocannabinol (THC) from a plant material, the process comprising extracting the THC from the plant material using supercritical CO₂ at a temperature >65° C.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIG. 1 is a graph that illustrates THC recovery data from extracting cannabis plant material with supercritical CO₂ as a function of temperature and/or pressure in accordance with an embodiment of the present disclosure.

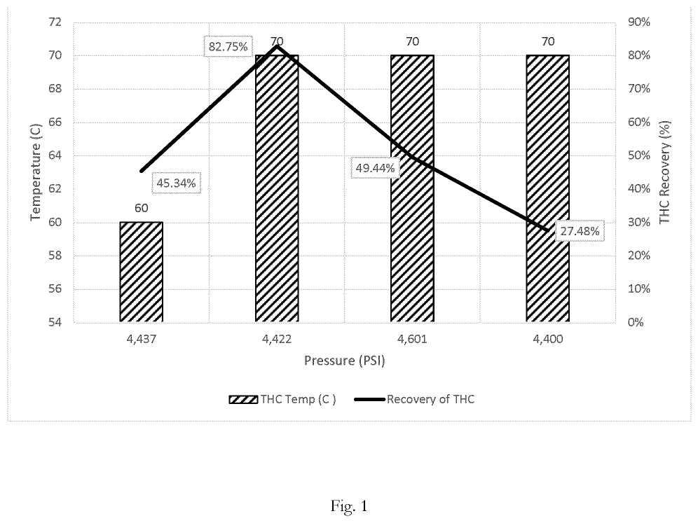

FIG. 2 is a graph that illustrates a THC recovery model for extracting cannabis plant material with supercritical CO₂ as a function of temperature and/or pressure in accordance with an embodiment of the present disclosure.

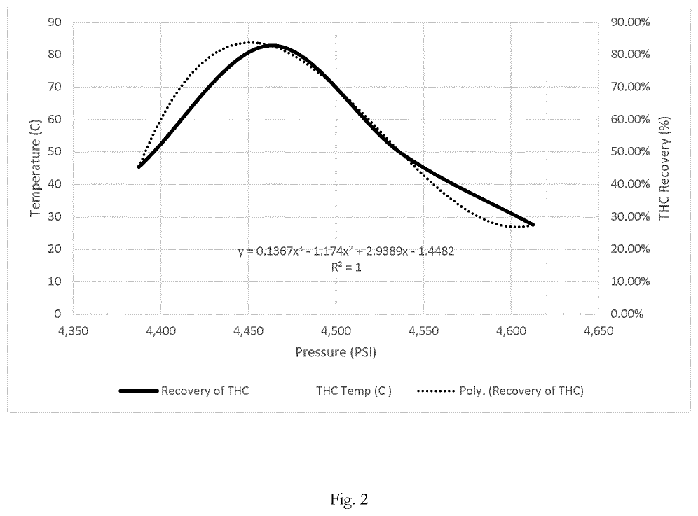

FIG. 3 is a graph that illustrates the CBD recovery data from extracting cannabis plant material with supercritical CO₂ as a function of temperature and/or pressure in accordance with an embodiment of the present disclosure.

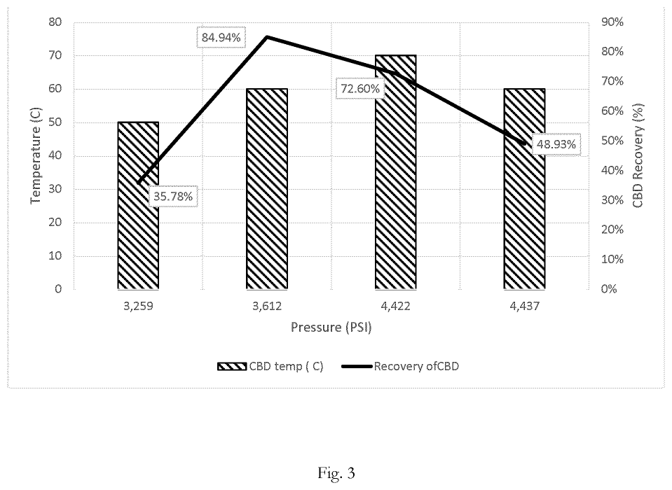

FIG. 4 is a graph that illustrates a CBD recovery model from extracting cannabis plant material with supercritical CO₂ as a function of temperature and/or pressure in accordance with an embodiment of the present disclosure.

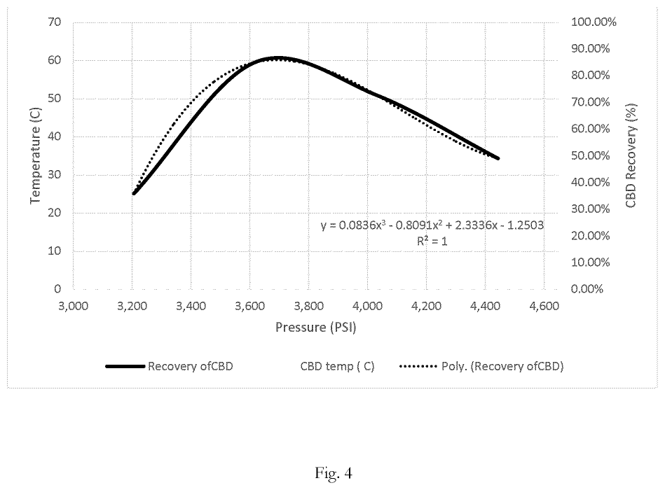

FIG. 5 is a graph that illustrates THC and CBD recovery data from extracting cannabis plant material with supercritical CO₂ as a function of temperature and/or pressure in accordance with an embodiment of the present disclosure.

FIG. 6 is a graph that illustrates THC recovery model from extracting cannabis plant material with supercritical CO₂ as a function of temperature at a fixed pressure in accordance with an embodiment of the present disclosure.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

The present inventor has through extensive R&D work surprisingly and unexpectedly discovered that one can vary supercritical CO₂ extraction parameters in order to control the selective extraction of tetrahydrocannabinol (THC) over cannabidiol (CBD), and vice versa, from cannabis plant material, where one is less constrained by the intrinsic THC and CBD levels present in the cannabis plant material. The present inventor thus proposes a cannabis concentration process to obtain a cannabis concentrate from cannabis plant material which is improved in comparison to known prior art methods. This has been unexpected and surprising especially since at least WO 2018/061009 explicitly teaches that supercritical CO₂ extraction is far from being selective for specific cannabinoids, and may concomitantly extract also various essential oils.

In one broad aspect, the present disclosure proposes a process for producing a cannabis concentrate from cannabis plant material where one can modulate the extraction levels of THC and CBD by varying the supercritical CO₂ extraction parameters in order to obtain a cannabis concentrate having a cannabinoid profile which is different from the cannabinoid profile in the plant materials.

Advantageously, such control over the selective extraction of THC and CBD over one another allows one to tailor and design the cannabis concentrate to desired cannabinoid levels/ratios, less constrained by the intrinsic cannabinoid levels/ratios present in the cannabis plant material. Furthermore, such process can enable use of low-quality cannabis strains to generate cannabis concentrate having desirable cannabinoid profiles, thereby, realizing cost savings in product manufacturing.

Optimization of extraction and fractionation conditions has been deemed difficult in the art due to the lack of fundamental thermodynamic solubility data and phase equilibria. Solubility data from the Delft study (Perrotin-Brunel et al., J. Chem. Eng. Data, 2010, 55 (9), pp 3704-3707) show that CBD and THC solubility increases with pressure, differentially for the major cannabinoid components allowing for their fractionation at pressures from 13.0 to 20.2 MPa (1885 to 2929 psi) and temperatures between 40-60° C. This Delft study concluded that “the cannabis plant was containing a lot of Δ9-THC compared to other cannabinoids, it was not possible to obtain a selective process to isolate one particular cannabinoid.” The present inventor believes that the Delft study failed to obtain selective extraction of THC over CBD, and vice versa, because the Delft study failed to realize that the low temperatures and low pressures used did not enable selective extraction of THC/CBD.

U.S. Pat. No. 9,044,390 teaches that supercritical fluid extraction can be used to selectively obtain one or more desired substances from the Cannabis plant material, while selectively excluding one or more undesired substances. However, U.S. Pat. No. 9,044,390 does not describe how to modulate the extraction levels of THC and CBD over one another by varying the supercritical CO₂ extraction parameters in order to obtain a cannabis concentrate having a given cannabinoid profile. In fact, in the experimental data provided in U.S. Pat. No. 9,044,390, THC and CBD are both extracted at the same time seemingly without any selective extraction.

WO 2016/187679 and US 2015/0297654 each describes varying supercritical CO₂ extraction pressure parameters in order to obtain a cannabis concentrate having a given cannabinoid profile. In WO 2016/187679, the CBD and THC temperatures are selected within the same range of 40-60° C., and THC selective pressures are selected within the range of 1885-3335 psi, while the CBD selective pressures are selected within the range of 1595-2465 psi. In US 2015/0297654, the CBD and THC temperatures are selected within the same range of 35-60° C., and THC selective pressures are selected within the range of 750-1500 psi, while the CBD selective pressures are selected within the range of 1500-3000 psi.

US 2018/0099236 describes varying supercritical CO₂ extraction pressure and temperature parameters in order to obtain a cannabis concentrate having a given cannabinoid profile. The THC temperatures are selected within the range of <55° C., and the THC selective pressures are selected within the range of 1100-2000 psi. The CBD temperatures are selected within the range of <60° C., and the CBD selective pressures are selected within the range of 2000-8000 psi.

In contrast, the herein described improved process is implemented by selecting a temperature that allows one to selectively extract THC over CBD, and vice versa, from a cannabis plant material. This improved process can also include selecting a pressure that further allows one to selectively extract THC over CBD, and vice versa, from a cannabis plant material. The resulting cannabis concentrate can, thus, be formulated “on-demand” and as per application, less constrained by the intrinsic cannabinoid levels/profile of the plant material.

For example, for a given application, one can select to perform the herein described process using supercritical CO₂ extraction parameters selected for controlling selective extraction of CBD over THC from the plant material for a first period of time, and then, for a second period of time, one can select supercritical CO₂ extraction parameters selected for controlling selective extraction of THC over CBD from the plant material for a second period of time. Once the extraction is concluded, one obtains a cannabis concentrate containing CBD and THC in a ratio that substantially corresponds to a ratio of the first time period to the second time period used. For instance, a first time period of 1 h using parameters selected for controlling selective extraction of CBD over THC and a second time period of 30 minutes using parameters selected for controlling selective extraction of THC over CBD will result in a 2:1 CBD:THC ratio, independently of the intrinsic CBD:THC ratio present in the plant material.

In one non-limiting embodiment, the herein described process makes use of supercritical CO₂ extraction to produce a cannabis concentrate including a first to second cannabinoid ratio (e.g., CBD to THC) selected in the range of between about 40:1 and about 1:40, including any values therein, for example: 1:35, 1:30, 1:25, 1:20, 1:15, 1:10, 1:5, 1:2, 1:1, 2:1, 5:1, 10:1, 15:1, 20:1, 25:1, 30:1, 35:1, 40:1, and the like.

In one non-limiting embodiment, the supercritical CO₂ extraction parameters are selected for controlling selective extraction of a first cannabinoid (e.g., CBD) from the plant material so as to obtain a cannabis concentrate which is at least selectively enriched in the first cannabinoid i.e., representing more than 51 wt. % of total cannabinoids, for example more than 60 wt. %, more than 70 wt. %, more than 80 wt. %, more than 85 wt. %, more than 90 wt. %, and the like.

In one non-limiting embodiment, the supercritical CO₂ extraction parameters are selected for controlling selective extraction of the second cannabinoid from the plant material so as to obtain a cannabis concentrate which is at least selectively enriched in the second cannabinoid (e.g., THC), i.e., representing more than 51 wt. % of total cannabinoids, for example more than 60 wt. %, more than 70 wt. %, more than 80 wt. %, more than 85 wt. %, more than 90 wt. %, and the like.

In one non-limiting embodiment, the supercritical CO₂ extraction parameters are selected for controlling selective extraction of the second cannabinoid from the plant material so as to obtain a cannabis concentrate which is at least selectively enriched in the desired cannabinoid (e.g., the first or the second cannabinoid discussed above), i.e., representing more than 51 wt. % of total extract, for example more than 60 wt. %, more than 70 wt. %, more than 80 wt. %, more than 85 wt. %, more than 90 wt. %, and the like.

In one non-limiting embodiment, the supercritical CO₂ extraction parameters for selectively extracting THC include a temperature of at least 65° C. For example, the supercritical CO₂ extraction parameters for selectively extracting THC may include a temperature selected in the range of 70-90° C., and the like. In one embodiment, the supercritical CO₂ extraction parameters for selectively extracting THC may further include a pressure of at least 4400 psi, for example 4500 psi, or 4600, or 5000 psi, and the like.

In one non-limiting embodiment, the supercritical CO₂ extraction parameters for selectively extracting CBD include a temperature of less than 65° C. For example, the supercritical CO₂ extraction parameters for selectively extracting CBD may include a temperature selected in the range of 40-60° C., and the like. In one embodiment, the supercritical CO₂ extraction parameters for selectively extracting CBD may further include a pressure of less than 4600 psi, for example selected in the range of between 2000 and 4600 psi, such as 3000 psi, or 3500 psi, or 4000 psi, and the like.

In a non-limiting practical implementation, the process for producing a cannabis concentrate containing a controlled ratio of CBD to THC from cannabis plant material may, thus, include selecting supercritical CO₂ extraction parameters for selectively controlling extraction of CBD in a first extraction stage and THC in a second extraction stage, and extracting the cannabis plant material with supercritical CO₂ under the selected parameters to obtain the extract containing the controlled ratio of CBD to THC.

The herein described cannabis concentrate can be used for mixing with other components, solvents, emulsifiers, carriers, and the like, for making various products intended for human consumption. Examples of products intended for human consumption are described for example in any one of 62/725,142, 62/719,942, 62/722,422, 62/719,966, 62/725,308 and 62/719,926, each of which is herein incorporated by reference herein in its entirety, and may include at least edibles, beverages, vaping oils for use in vaping devices, and the like.

For example, such product intended for human consumption can be a beverage such as a drink, including water or other liquid; or concentrates, powders, crystals and other mixes or substances which are primarily used to make drinks but are not alone intended to be consumed without adding water or some other liquid.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

For the purpose of this specification, the expression “CO₂ extraction” refers to a super fluid extraction process that can be performed in two ways: supercritical and subcritical extraction. When CO₂ is used as solvent, it has different characteristics which depend on its fluid state. Subcritical CO₂ defines CO₂ at the state between 5-10° C. (278.15-283.15 K, 41-50° F.) and a pressure of between 800-1500psi (54.43-102.06 atm, 5.51-10.24 MPa). At this temperature and pressure, CO₂ behaves as a thick fluid. When temperature and pressure conditions are increased and surpass the critical temperature (304.25 K, 31.10° C., 87.98° F.) and critical pressure (72.9 atm, 7.39 MPa, 1,071 psi), the CO₂ expands in the container like a gas but with a density like that of a liquid. This is known as supercritical carbon dioxide (sCO₂ or SC—CO₂). Subcritical CO₂ extraction uses low temperature and low pressure and thus takes more time. Subcritical CO₂ extraction gives smaller yields and can might retain some terpenes and oils. For supercritical CO₂ extraction, higher temperatures and higher pressures are applied, which can damage terpenes and other phytochemicals

For the purpose of this specification, the term “cannabis” refers to a genus of flowering plants that includes three different species, Cannabis sativa, Cannabis indica and Cannabis ruderalis. The term “cannabis plant(s)” encompasses wild type cannabis and also variants thereof, including cannabis chemovars which naturally contain different amounts of the individual cannabinoids. For example, some cannabis strains have been bred to produce minimal levels of THC, the principal psychoactive constituent responsible for the high associated with it and other strains have been selectively bred to produce high levels of THC and other psychoactive cannabinoids.

For the purpose of this specification, the expression “cannabinoid” means a compound such as cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin(THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2, 6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

For the purpose of this specification, the expressions “cannabidiol” or “CBD” are generally understood to refer to one or more of the following compounds, and, unless a particular other stereoisomer or stereoisomers are specified, includes the compound “Δ2-cannabidiol.” These compounds are: (1) A5-cannabidiol (2-(6-isopropenyl-3-methyl-5-cyclohexen-1-yl)-5-pentyl-1,3-benzenediol); (2) A4-cannabidiol (2-(6-isopropenyl-3-methyl-4-cyclohexen-1-yl)-5-pentyl-1,3-benzenediol); (3) A3-cannabidiol (2-(6-isopropenyl-3-methyl-3-cyclohexen-1-yl)-5-pentyl-1,3-benzenediol); (4) A3,7-cannabidiol (2-(6-isopropenyl-3-methylenecyclohex-1-yl)-5-pentyl-1,3-benzenediol); (5) A2-cannabidiol (2-(6-isopropenyl-3-methyl-2-cyclohexen-1-yl)-5-pentyl-1,3-benzenediol); (6) A1-cannabidiol (2-(6-isopropenyl-3-methyl-1-cyclohexen-1-yl)-5-pentyl-1,3-benzenediol); and (7) A6-cannabidiol (2-(6-isopropenyl-3-methyl-6-cyclohexen-1-yl)-5-pentyl-1,3-benzenediol).

### EXAMPLES

It should be understood that these examples are for illustrative purposes only and are not meant to limit the scope of the compositions and processes described herein.

The inventor hypothesized that the solubility of THC in supercritical CO₂ fluid is dependent on pressure, temperature and time.

Extensive R&D work was initiated to test these hypotheses by solving a modified Peng-Robinson equation of state, in which the main operating variable is pressure. There are several unknown properties of THC and CBD that are needed to solve for the solubility of THC/CBD in supercritical CO₂. This required experimental design to empirically determine the effects of the variables (T,P,V,t) on the solubility of THC/CBD, which ultimately translates into yields. The first set of a Design of Experiments (DOE) was a 5-level factorial design. Using the results from the 5-level work, a second set of experiments was conducted. This was a 2-level factorial to focus on the pressure and flow rate. The temperature was set at 60° C. for these runs.

The work attempts to test these hypotheses by solving the modified Peng-Robinson equation of state:

(P+aav^{˜}2+2bv^{˜}−b2)(v^{˜}−b)=RT

Since the proposed main operating variable is pressure the equation becomes:

P=RTv^{˜}−b−aav^{˜}2+2bv^{˜}−b2

Coefficients “a” and “b” are made functions of the critical properties by imposing the criticality conditions.

a=[1+(0.37464+1.54226ω−0.26992ω2)(√1−Tr)]²

a=0.45724R2T2cPc

b=0.07780RTcPc

There are several unknown physical properties of THC and CBD. These are needed to be able to solve for the solubility of THC/CBD in supercritical CO₂. The first set of DOE was a 5 level factorial design (Tables 1 and 2).

TABLE 1
5 level factorial design

| CV | run1 | run2 | run3 | run4 | run5 | run6 | run7 |
|---|---|---|---|---|---|---|---|
| P | h | H | H | h | h | l | L |
| T | h | H | H | h | l | l | H |
| V | h | H | H | l | l | l | L |
| t | h | H | L | l | l | L | L |
| P [symbol] | h | L | l | l | l | L | L |

TABLE 2

| Control variable | High | Low |
|---|---|---|
| Pressure (psi) | 4000 | 2000 |
| Temperature (C.) | 70 | 50 |
| Volumetric flow rate (ml/min | 300 | 200 |
| Time of extraction (hours) | 2 | 1 |
| Packing density (heuristic) | Densely | Loosely |
| | packed | packed |

Using the results from the 5-level work, a second set of experiment was conducted. This was a two-level factorial to focus on the pressure and flow rate (Tables 3, 4 and 5).

TABLE 3
2 level factorial design at 60° C.

| T = 60° C. | | | | |
|---|---|---|---|---|
| CV | run1 | run2 | run3 | run4 |
| P | H | H | L | L |
| V | H | L | H | L |
| bag condition | Iced | Iced | Waxy | Waxy |
| % recovery | 10 | 8 | 6 | 6 |
| operation notes | hard, | easy, low | hard, CO₂ | easy no |
| | overpressure | yield in | ice buildup, | CO₂ ice |
| | risk | batch-1 | ppm = 8000 | on exhaust |

TABLE 4
2 level at 70° C.

| T = 70° C. | | | | |
|---|---|---|---|---|
| 2 lvl = 4 runs | run1 | run2 | run3 | run4 |
| P | h | H | L | L |
| V | h | L | H | L |

From the extraction data, the inventor was able to empirically establish the dependence of THC/CBD solubility on the operating pressure and temperature. With increase in temperature for a given pressure (i.e., in supercritical conditions) the extraction efficiency of bulk cure resin also increased. Also an improvement on the w/w extraction of THC/CBD was also observed.

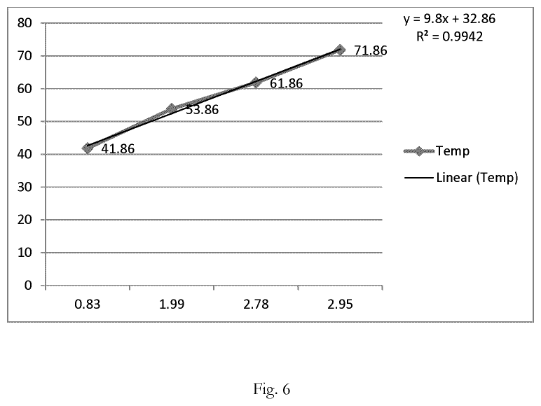

The increase in temperature to 80° C. and 90° C. was calculated using this model and it predicted a cannabis concentration of cannabinoids to be 83.1% at 80° C. and 89.6% at 90° C. The purity of the extract would be 63.3% THC at 80° C. and 69.8% THC at 90° C. The model was correlated to an R-value of 0.99 (Table 5 and FIG. 6).

TABLE 5
Extraction model

γ at 23.3 MPa
(3,379 psi)

| T (° K) | T (° C.) | (g) | (%) |
|---|---|---|---|
| 315 | 41.86 | 0.83 | |
| 327 | 53.86 | 1.99 | 139.76 |
| 335 | 61.86 | 2.78 | 39.70 |
| 345 | 71.86 | 2.95 | 6.12 |
| | 80 | 3.13 | 6.12 |
| | 90 | 3.32 | 12.60 |
| Calculated | 80 | [THC] | 63.3 |
| Calculated | | [C] | 83.1 |
| Calculated | 90 | [THC] | 69.8 |
| Calculated | | [C] | 89.6 |

The model was adjusted by 30% in an attempt to predict the effects of higher flow rate. This would be an increased from the 300 ml/min to 600 ml/min.

TABLE 6
Extraction model 30% adjustment

| | |
|---|---|
| | γ at 23.3 MPa |
| Calculated | (3,379 psi) |

| temp (° C.) | (g) | (%) |
|---|---|---|
| 80 | 3.37 | 14.14% |
| 90 | 4.08 | 30.38% |
| 80 | [THC] | 71.34% |
| | [Cannabinoids] | 91.1% |
| 90 | [THC] | 87.58% |
| | [Cannabinoids] | 107.38% |

The same experiments were performed but using a first set of parameters for controlling selective extraction of CBD, and a second set of parameters for controlling selective extraction of THC so as to obtain a cannabis concentrate containing a controlled THC to CBD ratio (table 7).

The results in table 7 were obtained from a cannabis plant material having an initial CBD:THC ratio of about 1.48 (3.88% THC and 5.76% CBD).

TABLE 7
Selective extraction of cannabinoids from a cannabis plant
material having an initial CBD:THC ratio of about 1.48

| | Extract16 | Extract17 | Extract18 | Extract19 |
|---|---|---|---|---|
| CBD temp (° C.) | 60 | 70 | 60 | 50 |
| CBD pressure (psi) | 4,437 | 4,422 | 3,612 | 3,259 |
| THC temp (° C.) | 60 | 70 | 70 | 70 |
| THC pressure (psi) | 4,437 | 4,422 | 4,601 | 4,400 |
| Extracted [THC] (g) | 34.4 | 76.45 | 41.15 | 28.00 |
| Extracted [CBD] (g) | 40.3 | 116.5 | 89.7 | 74.00 |
| crude Extract weight (g) | 74.7 | 192.95 | 130.85 | 102 |
| [THC] in the extracted | 46.05 | 39.62 | 31.45 | 27.45 |
| concentrate (%) | | | | |
| [CBD] in the extracted | 53.95 | 60.38 | 68.55 | 72.55 |
| concentrate (%) | | | | |
| Cannabis processed (g) | 1,955.40 | 2,381.20 | 2,145.00 | 2,625.90 |
| Available THC (g) | 75.87 | 92.39 | 83.23 | 101.88 |
| Available CBD (g) | 112.63 | 137.16 | 123.55 | 151.25 |
| Recovery THC (%) | 45.34 | 82.75 | 49.44 | 27.48 |
| Recovery CBD (%) | 35.78 | 84.94 | 72.60 | 48.93 |
| CBD:THC ratio | 1.17 | 1.52 | 2.18 | 2.64 |

From these experiments, it is clear that a temperature below 65° C. (e.g., 60° C.) in supercritical conditions, favors selective CBD extraction allowing one to obtain selective CBD:THC ratio extraction independently from the initial CBD:THC ratio present in the cannabis plant material.

It was observed that the previously discussed model for predicting solubility of THC/CBD in supercritical CO₂ is effective on the scaled-up system. As the flowrate was increased more cannabinoids were recovered. This was also observed in the weight lost in the post extraction material. The increase in the extraction chamber temperature also resulted in an increase in yield.

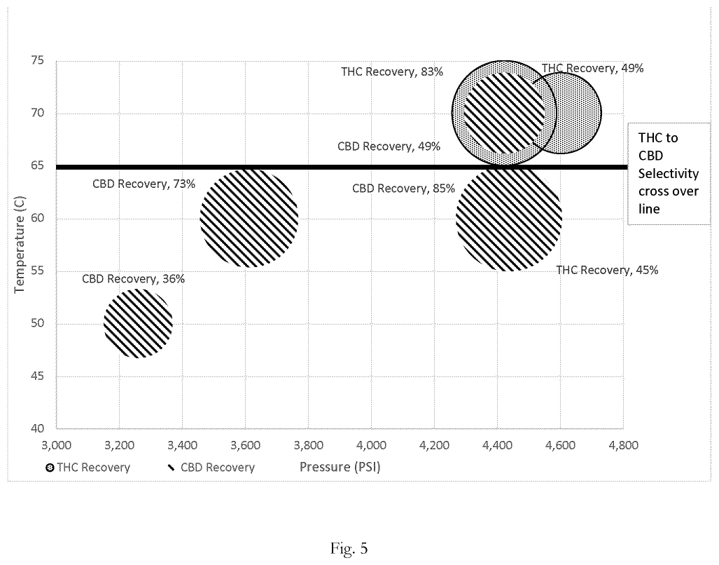

It was found that a maximum flow rate of 600 ml/min produced the crudest extract. Also, as shown in FIG. 5, it was found that the supercritical CO₂ extraction parameters THC to CBD selectivity cross over line is at 65° C. In other words, so long as the temperature is maintained below 65° C., the extraction process with supercritical CO₂ will selectively extract CBD over THC from the cannabis plant material. Conversely, when the temperature is maintained above 65° C., the extraction process with supercritical CO₂ will selectively extract THC over CBD from the cannabis plant material.

In one embodiment, it was found that a higher temperature above 65° C., preferably selected in the range of 70-90° C., produces better results for extracting THC. Additionally, when the supercritical CO₂ extraction parameters also include a pressure of at least 4200 psi, it was found that the extraction process further selectively extracts THC over CBD from the plant material.

In one embodiment, it was found that a temperature below 65° C., preferably selected in the range of 40-60° C. produces better results for extracting CBD. Additionally, when the supercritical CO₂ extraction parameters also include a pressure of less than 4600 psi, for example selected in the range of 2000 and 4600 psi, it was found that the extraction process further selectively extracts CBD over THC from the plant material.

In one embodiment, it was shown that extraction of cannabis plant material in supercritical CO₂ at 5000 psi and 90° C. for 1 h produced a THC-rich concentrate, whereas extraction in supercritical CO₂ at 3000 psi and 50° C. for 1 h produced a CBD-rich concentrate, independently of the initial THC/CBD levels or ratio in the plant material.

Furthermore, the results obtained confirm that implementation of this extraction process resulted in an increase (compared to known prior art methods) in cannabis concentrate recovery of 20% on a weight of concentrate collected vs weight of cannabis used (w/w).

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Unless otherwise defined, all technical and scientific terms used herein have the same meaning as commonly understood by a person of ordinary skill in the art to which the present invention pertains.

Although various embodiments of the disclosure have been described and illustrated, it will be apparent to those skilled in the art in light of the present description that numerous modifications and variations can be made. The scope of the invention is defined more particularly in the appended claims.

1. A process for extracting a cannabis concentrate from a cannabis plant material, the process comprising
providing the plant material, the plant material having a first cannabinoid profile, and
extracting the cannabis concentrate from the plant material using supercritical CO2, the cannabis concentrate having a second cannabinoid profile wherein the first and second profiles are different, wherein the extracting includes performing supercritical CO₂ extraction at a temperature >65° C. for selectively extracting tetrahydrocannabinol (THC) over cannabidiol (CBD) from the plant material.

2. The process of claim 1, wherein the extracting is performed at a temperature selected in the range of 70 to 90° C. for selectively extracting THC over CBD from the plant material.

3. The process of claim 1, wherein the extracting is performed at a pressure of ≥4200 psi for selectively extracting THC over CBD from the plant material.

4. The process of claim 3, wherein the extracting is performed at a pressure of ≤5000 psi for selectively extracting THC over CBD from the plant material.

5. The process of claim 1, wherein the extracting further includes performing supercritical CO₂ extraction at a temperature of <65° C. for selectively extracting CBD over THC from the plant material.

6. The process of claim 5, wherein the extracting is performed at a temperature selected in the range of 40 to 60° C. for selectively extracting CBD over THC from the plant material.

7. The process of claim 5, wherein the extracting is performed at a pressure of ≤4600 psi for selectively extracting CBD over THC from the plant material.

8. The process of claim 7, wherein the extracting is performed at a pressure selected in the range of 2000 and 4600 psi for selectively extracting CBD over THC from the plant material.

9. The process of claim 1, comprising a first extraction step for selectively extracting CBD over THC from the plant material, and a second extraction step for selectively extracting THC over CBD from the plant material.

10. A process for extracting tetrahydrocannabinol (THC) from a plant material, the process comprising extracting the THC from the plant material using supercritical CO₂ at a temperature >65° C.

11. The process of claim 10, wherein the extracting is performed at a temperature selected in the range of 70 to 90° C.

12. The process of claim 10, wherein the extracting is performed at a pressure of ≥4200 psi.

13. The process of claim 12, wherein the extracting is performed at a pressure of 5000 psi.
